(19) United States
(12) Patent Application Publication
(10) Pub. No.: US 2013/0195877 A1
(43) Pub. Date: Aug. 1, 2013

(54) TREATMENT OF CACHEXIA BY TARGETING INTERLEUKIN-1 BETA

(71) Applicant: XBiotech, Inc., Vancouver (CA)

(72) Inventor: John Simard, Austin, TX (US)

(73) Assignee: XBiotech, Inc., Vancouver (CA)

(21) Appl. No.: 13/751,396

(22) Filed: Jan. 28, 2013

Related U.S. Application Data
(60) Provisional application No. 61/592,823, filed on Jan. 31, 2012.

(51) Int. Cl. C07K 16/24

(52) U.S. Cl. CPC C07K 16/245
USPC 424/139.1

(57) ABSTRACT

Administration of a mAb that specifically binds IL-1β is used for treating cachexia in human subjects.

### CROSS-REFERENCE TO RELATED APPLICATIONS

This application claims priority from U.S. patent application Ser. No. 61/592,823 filed on Jan. 31, 2012.

### STATEMENT AS TO FEDERALLY SPONSORED RESEARCH

Not applicable.

### FIELD OF THE INVENTION

The invention relates generally to the fields of medicine, oncology, and immunology. More particularly, the invention relates to the use of agents such as antibodies (Abs) which specifically bind interleukin-1β(IL-1β) to treat one or more symptoms of cachexia.

### BACKGROUND

Cachexia is a condition characterized by weight loss, muscle atrophy, anorexia, fatigue, and weakness. It is commonly seen in patients with chronic progressive diseases such as AIDS, hormone deficiency, chronic obstructive lung disease (COPD), congestive heart failure (CHF), tuberculosis (TB), and cancer. In cachexia, a decline in food intake relative to energy expenditure leads to weight loss. Even with adequate nutritional support, abnormalities in the metabolism of carbohydrates, proteins, and fats causes continued mobilization and ineffective repletion of host lean (muscle) tissue. The physiological mechanisms that cause cachexia remain poorly understood, although cachectin/TNF or other inflammatory cytokines have been implicated.

Nutritional support can help maintain the body weight of cachectic patients, but does not prevent loss of lean muscle mass. Steroids (particularly progesterone-like drugs) can increase appetite and reverse weight loss, although again there is no evidence that it reverses the loss of muscle mass. A humanized anti-IL-6 antibody was evaluated as a treatment for cachexia in a clinical trial involving lung cancer patients. The antibody appeared to be safe and well tolerated, improved the lung symptom score, reversed fatigue, and reduced the rate of lean body mass loss. However, muscle loss as a result of cachexia was largely unabated.

### SUMMARY

The invention relates to agents that specifically target IL-1β for improving the symptoms of cachexia in human patients—including slowing, halting or completely reversing the loss of lean body mass.

Accordingly, the invention features a method of treating cachexia in a human subject by administering to the subject a pharmaceutical composition including a pharmaceutically acceptable carrier and an amount of an IL-1β-targeting agent effective to ameliorate a symptom of cachexia. The IL-1β-targeting agent can be an anti-IL-1β Ab such as an anti-IL-1β monoclonal Ab (mAb).

The pharmaceutical composition can be administered to the subject by injection, subcutaneously, intravenously, or intramuscularly. In the method, the dose administered to the patient can be at least 0.05 (e.g., at least 0.10, 0.25, 0.5, 0.75., 1, 2, 3, 4, 5, 10 or 20) mg/kg of body weight.

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of medical terms can be found in Stedman's Medical Dictionary, 27^{th} Edition, Lippincott, Williams & Wilkins, 2000.

As used herein, an “Ab” or “Ab” is an immunoglobulin (Ig), a solution of identical or heterogeneous Igs, or a mixture of Igs. An “Ab” can also refer to fragments and engineered versions of Igs such as Fab, Fab′, and F(ab′)₂ fragments; and scFv's, heteroconjugate Abs, and similar artificial molecules that employ Ig-derived CDRs to impart antigen specificity. A “mAb” or “mAb” is an Ab expressed by one clonal B cell line or a population of Ab molecules that contains only one species of an antigen binding site capable of immunoreacting with a particular epitope of a particular antigen. A “polyclonal Ab” or “polyclonal Ab” is a mixture of heterogeneous Abs. Typically, a polyclonal Ab will include myriad different Ab molecules which bind a particular antigen with at least some of the different Abs immunoreacting with a different epitope of the antigen. As used herein, a polyclonal Ab can be a mixture of two or more mAbs.

An “antigen-binding portion” of an Ab is contained within the variable region of the Fab portion of an Ab and is the portion of the Ab that confers antigen specificity to the Ab (i.e., typically the three-dimensional pocket formed by the CDRs of the heavy and light chains of the Ab). An “Fab portion” or “Fab region” is the proteolytic fragment of a papain-digested Ig that contains the antigen-binding portion of that Ig. A “non-Fab portion” is that portion of an Ab not within the Fab portion, e.g., an “Fc portion” or “Fc region.” A “constant region” of an Ab is that portion of the Ab outside of the variable region. Generally encompassed within the constant region is the “effector portion” of an Ab, which is the portion of an Ab that is responsible for binding other immune system components that facilitate the immune response. Thus, for example, the site on an Ab that binds complement components or Fc receptors (not via its antigen-binding portion) is an effector portion of that Ab.

When referring to a protein molecule such as an Ab, “purified” means separated from components that naturally accompany such molecules. Typically, an Ab or protein is purified when it is at least about 10% (e.g., 9%, 10%, 20%, 30% 40%, 50%, 60%, 70%, 80%, 90%, 95%, 98%, 99%, 99.9%, and 100%), by weight, free from the non-Ab proteins or other naturally-occurring organic molecules with which it is naturally associated. Purity can be measured by any appropriate method, e.g., column chromatography, polyacrylamide gel electrophoresis, or HPLC analysis. A chemically-synthesized protein or other recombinant protein produced in a cell type other than the cell type in which it naturally occurs is “purified.”

By “bind”, “binds”, or “reacts with” is meant that one molecule recognizes and adheres to a particular second molecule in a sample, but does not substantially recognize or adhere to other molecules in the sample. Generally, an Ab that “specifically binds” another molecule has a K_{d} greater than about 10⁶, 10⁷, 10⁸, 10⁹, 10¹⁰, 10¹¹, 10¹² or 10¹³ liters/mole for that other molecule.

A “therapeutically effective amount” is an amount which is capable of producing a medically desirable effect in a treated animal or human (e.g., amelioration or prevention of a disease or symptom of a disease).

Although methods and materials similar or equivalent to those described herein can be used in the practice or testing of the present invention, suitable methods and materials are described below. All applications and publications mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention encompasses compositions and methods for ameliorating one or more symptoms of cachexia in a subject. The below described preferred embodiments illustrate adaptation of these compositions and methods. Nonetheless, from the description of these embodiments, other aspects of the invention can be made and/or practiced based on the description provided below.

### General Methodology

Methods involving conventional immunological and molecular biological techniques are described herein. Immunological methods (for example, assays for detection and localization of antigen-Ab complexes, immunoprecipitation, immunoblotting, and the like) are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Ab methods are described in Handbook of Therapeutic Abs, Dubel, S., ed., Wiley-VCH, 2007. General methods of medical treatment are described in McPhee and Papadakis, Current Medical Diagnosis and Treatment 2010, 49^{th} Edition, McGraw-Hill Medical, 2010; and Fauci et al., Harrison's Principles of Internal Medicine, 17^{th} Edition, McGraw-Hill Professional, 2008

### Treatment of Cachexia

The compositions and methods described herein are useful for treating cachexia in a mammalian subject by administering to the subject a pharmaceutical composition including an amount of an IL-1β-targeting agent effective to improve at least one characteristic of cachexia in the subject. The mammalian subject might be any that suffers from cachexia including, human beings, dogs, cats, horses, cattle, sheep, goats, and pigs. Human subjects might be male, female, adults, children, or seniors (65 and older). The mammalian subject can be one with cancer (particularly metastatic cancer, solid tumor cancers, and stage II-IV cancers), HIV infection, TB, COPD, CHF, chronic renal failure, a hormone imbalance, severe trauma (e.g., burns), hypermetabolism (e.g., sustained elevated heart rate of at least 6 bpm over normal for a given subject), excessive sympathetic nerve activity, a hyper-inflammatory state (e.g., elevated CRP levels, increased IL-6 levels, increased TNFβ levels, and/or increased IFNγ levels), a>51b weight loss in the preceding 2 months, and/or an estimated daily caloric intake of <20 cal/kg. The subject may also be on that is being or has been treated with steroids, nutritional supplements, and/or appetite stimulants.

Any symptom of cachexia susceptible to amelioration by administration of an IL-1β targeting agent might be targeted. Examples of such symptoms include weakness, fatigue, gastrointestinal distress, sleep/wake disturbances, pain, listlessness, shortness of breath, lethargy, depression, malaise, anorexia, weight loss, muscle atrophy, and loss lean body mass. The improvement, if measureable by percent, can be at least 1, 2, 3, 4, 5, 10, 15, 20, 25, 30, 40, 50, 60, 70, 80, or 90%. Symptoms such as weakness, fatigue, pain, listlessness, depression, and malaise can be measured by techniques known in the art (e.g., using tests such as EORTC-global quality of life, the Beck Depression Inventory, the Zung Self-rating Depression Scale, the Center for Epidemiologic Studies-Depression Scale, the Hamilton Rating Scale for Depression, and patient self-reporting). For assessing anorexia, muscle mass, or lean body mass assessment, dual-emission X-ray absorptiometry scan (DEXA), bioelectrical impedance analysis (BIA), indirect calorimetry, nutrition diaries, and similar known methods can be used.

### Antibodies and other Agents that Target IL-1β

Any suitable type of Ab or other biologic agent (e.g., a fusion protein including an IL-1β-binding component such as an IL-1 receptor) that specifically binds IL-1β and reduces a characteristic of cachexia in a subject might be used in the invention. For example, the anti-IL-1β Ab used might be mAb, a polyclonal Ab, a mixture of mAbs, or an Ab fragment or engineered Ab-like molecule such as an scFv. The Ka of the Ab is preferably at least 1×10⁹ M⁻¹ or greater (e.g., greater than 9×10¹⁰ M⁻, 8×10¹⁰ M⁻, 7×10¹⁰ M⁻¹, 6×10¹⁰ M⁻¹, 5×10¹⁰ M⁻¹, 4×10¹⁰ M⁻¹, 3×10¹⁰ M⁻¹, 2×10¹⁰ M⁻¹, or 1×10¹⁰ M⁻¹). In a preferred embodiment, the invention utilizes a fully human mAb that includes (i) an antigen-binding variable region that exhibits very high binding affinity (e.g., at least nano or picomolar) for human IL-1β and (ii) a constant region. The human Ab is preferably an IgG1, although it might be of a different isotype such as IgM, IgA, or IgE, or subclass such as IgG2, IgG3, or IgG4.

Because B lymphocytes which express Ig specific for human cytokines occur naturally in human beings, a presently preferred method for raising mAbs is to first isolate such a B lymphocyte from a subject and then immortalize it so that it can be continuously replicated in culture. Subjects lacking large numbers of naturally occurring B lymphocytes which express Ig specific for human IL-1β may be immunized with one or more human IL-1β antigens to increase the number of such B lymphocytes. Human mAbs are prepared by immortalizing a human Ab secreting cell (e.g., a human plasma cell). See, e.g., U.S. Pat. No. 4,634,664.

In an exemplary method, one or more (e.g., 5, 10, 25, 50, 100, 1000, 5000 or more) human subjects are screened for the presence of such human IL-1β-specific Ab in their blood. Those subjects that express the desired Ab can then be used as B lymphocyte donors. In one possible method, peripheral blood is obtained from a human donor that possesses B lymphocytes that express human IL-1β-specific Ab. Such B lymphocytes are then isolated from the blood sample, e.g., by cells sorting (e.g., fluorescence activated cell sorting, “FACS”; or magnetic bead cell sorting) to select B lymphocytes expressing human IL-1β-specific Ig. These cells can then be immortalized by viral transformation (e.g., using EBV) or by fusion to another immortalized cell such as a human myeloma according to known techniques. The B lymphocytes within this population that express Ig specific for human IL-1β can then be isolated by limiting dilution methods (e.g., cells in wells of a microtiter plate that are positive for Ig specific for human IL-1β are selected and subcultured, and the process repeated until a desired clonal line can be isolated). See, e.g., Goding, Monoclonal Antibodies: Principles and Practice, pp. 59-103, Academic Press, 1986. Those clonal cell lines that express Ig having at least nanomolar or picomolar binding affinities for human IL-1β are preferred. MAbs secreted by these clonal cell lines can be purified from the culture medium or a bodily fluid (e.g., ascites) by conventional Ig purification procedures such as salt cuts, size exclusion, ion exchange separation, and affinity chromatography.

Although immortalized B lymphocytes might be used in in vitro cultures to directly produce mAbs, in certain cases it might be desirable to use heterologous expression systems to produce mAbs. See, e.g., the methods described in U.S. patent application Ser. No. 11/754,899. For example, the genes encoding an mAb specific for human IL-1β might be cloned and introduced into an expression vector (e.g., a plasmid-based expression vector) for expression in a heterologous host cell (e.g., CHO cells, COS cells, myeloma cells, and E. coli cells). Because Igs include heavy (H) and light (L) chains in an H₂L₂ configuration, the genes encoding each may be separately isolated and expressed in different vectors.

Although generally less preferred due to the greater likelihood that a subject will develop an anti-Ab response, chimeric mAbs (e.g., “humanized” mAbs), which are antigen-binding molecules having different portions derived from different animal species (e.g., variable region of a mouse Ig fused to the constant region of a human Ig), might be used in the invention. Such chimeric Abs can be prepared by methods known in the art. See, e.g., Morrison et al., Proc. Nat'l. Acad. Sci. USA, 81:6851, 1984; Neuberger et al., Nature, 312:604, 1984; Takeda et al., Nature, 314:452, 1984. Similarly, Abs can be humanized by methods known in the art. For example, mAbs with a desired binding specificity can be humanized by various vendors or as described in U.S. Pat. Nos. 5,693,762; 5,530,101; or 5,585,089.

The mAbs described herein might be affinity matured to enhance or otherwise alter their binding specificity by known methods such as VH and VL domain shuffling (Marks et al. Bio/Technology 10:779-783, 1992), random mutagenesis of the hypervariable regions (HVRs) and/or framework residues (Barbas et al. Proc Nat. Acad. Sci. USA 91:3809-3813, 1994; Schier et al. Gene 169:147-155, 1995; Yelton et al. J. Immunol. 155:1994-2004, 1995; Jackson et al., J. Immunol. 154(7):3310-9, 1995; and Hawkins et al, J. Mol. Biol. 226:889-896, 1992. Amino acid sequence variants of an Ab may be prepared by introducing appropriate changes into the nucleotide sequence encoding the Ab. In addition, modifications to nucleic acid sequences encoding mAbs might be altered (e.g., without changing the amino acid sequence of the mAb) for enhancing production of the mAb in certain expression systems (e.g., intron elimination and/or codon optimization for a given expression system). The mAbs described herein can also be modified by conjugation to another protein (e.g., another mAb) or non-protein molecule. For example, a mAb might be conjugated to a water soluble polymer such as polyethylene glycol or a carbon nanotube (See, e.g., Kam et al., Proc. Natl. Acad. Sci. USA 102: 11600-11605, 2005). See, U.S. patent application Ser. No. 11/754,899.

Preferably, to ensure that high titers of human IL-1β-specific mAb can be administered to a subject with minimal adverse effects, the mAb compositions of the invention are at least 0.5, 1, 2, 3, 4, 5, 6, 7, 8, 9, 10, 11, 12, 13, 14, 15, 20, 25, 30, 35, 40, 45, 50, 60, 70, 80, 90, 95, 96, 97, 98, 99, 99.9 or more percent by weight pure (excluding any excipients). The mAb compositions of the invention might include only a single type of mAb (i.e., one produced from a single clonal B lymphocyte line) or might include a mixture of two or more (e.g., 2, 3, 4, 5, 6, 7, 8, 9, 10 or more) different types of mAbs.

While the IL-1β specific Abs described above are preferred for use in the invention, in some cases, other agents that specifically target IL-1β might be used so long as their administration leads to improvement of a characteristic of cachexia. These other agents might include small organic molecules, aptamers, peptides, and proteins that specifically bind IL-1β (e.g., anakinra or rilonacept). In addition, a vaccine that induces the production of IL-1β specific Abs might be used (e.g., IL-1β mixed with an adjuvant and/or conjugated with a carrier molecule can be administered to a subject once, twice, thrice or more times).

### Pharmaceutical Compositions and Methods

The anti-IL-1β Ab compositions may be administered to animals or humans in pharmaceutically acceptable carriers (e.g., sterile saline) that are selected on the basis of mode and route of administration and standard pharmaceutical practice. A list of pharmaceutically acceptable carriers, as well as pharmaceutical formulations, can be found in Remington's Pharmaceutical Sciences, a standard text in this field, and in USP/NF. Other substances may be added to the compositions and other steps taken to stabilize and/or preserve the compositions, and/or to facilitate their administration to a subject.

For example, the Ab compositions might be lyophilized (see Draber et al., J. Immunol. Methods. 181:37, 1995; and PCT/US90/01383); dissolved in a solution including sodium and chloride ions; dissolved in a solution including one or more stabilizing agents such as albumin, glucose, maltose, sucrose, sorbitol, polyethylene glycol, and glycine; filtered (e.g., using a 0.45 and/or 0.2 micron filter); contacted with beta-propiolactone; and/or dissolved in a solution including a microbicide (e.g., a detergent, an organic solvent, and a mixture of a detergent and organic solvent.

The Ab compositions may be administered to animals or humans by any suitable technique. Typically, such administration will be parenteral (e.g., intravenous, subcutaneous, intramuscular, or intraperitoneal introduction). The compositions may also be administered directly to a target site by, for example, injection. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

A therapeutically effective amount is an amount which is capable of producing a medically desirable result in a treated animal or human. An effective amount of anti-IL-1β Ab compositions is an amount which shows clinical efficacy in patients as measured by the improvement in one or more cachexia characteristics described above. As is well known in the medical arts, dosage for any one animal or human depends on many factors, including the subject's size, body surface area, age, the particular composition to be administered, sex, time and route of administration, general health, and other drugs being administered concurrently. Preferred doses range from about 0.2 to 20 (e.g., 0.05, 0.10, 0.15, 0.2, 0.3, 0.4, 0.5, 1, 2, 3, 4, 5, 6, 7, 8, 9, 10, 15, 20, or 40) mg/kg body weight. The dose may be given repeatedly, e.g., hourly, daily, semi-weekly, weekly, bi-weekly, tri-weekly, or monthly. Preferably 2 or more (e.g., 3, 4, 5, 6, 7, 8, 9, 10 or more) doses are given.

### EXAMPLES

### Example 1

A monoclonal IgG antibody that specifically binds IL-1β is prepared as a sterile injectable liquid formulation of 15 mg/mL antibody 1 in a stabilizing isotonic buffer (pH 6.4). Each 10-mL Type I borosilicate glass serum vial contains 4, 5 or 10 mL of the formulation, and is sealed with a 20-mm Daikyo Flurotec butyl rubber stopper and flip-off aluminum seal. The product is stored at 5±3° C., with excursions to room temperature permitted. The exact composition of the drug product is shown below:

Composition of the Drug Product

| Ingredient | Grade | Manufacturer | Concentration |
|---|---|---|---|
| IL-1β-specific IgG mAb | GMP | XBiotech | 15 mg/mL |
| sodium phosphate dibasic | compendial | JT Baker | 12 mg/mL |
| citric acid monohydrate | compendial | JT Baker | 2 mg/mL |
| Trehalose•2H2O | compendial | Ferro- | 60 mg/mL |
| (high-purity low | | Pfanstiehl | |
| endotoxin) | | | |
| polysorbate 80 | compendial | JT Baker | 0.2 mg/mL |
| Phosphoric acid, | compendial | JT Baker | 0.04 mg/mL |
| to adjust pH | | | |
| water for injection | compendial | Microbix | q.s. |

### Method of Administration

The calculated volume is withdrawn from the drug (mAb)-containing vial(s) using a suitable syringe. The drug is then injected into a small IV bag containing 100 mL of normal saline (0.9% NaCl) and mixed by inversion. The diluted drug product can be stored at room temperature for 3 hours prior to administration and is infused over a 1-hour period, with the subject being monitored for signs of an infusion reaction. The infusion is chased with a minimum of 30 mLs of normal saline to deliver any product that may be held up in the infusion set.

### Example 2

### Improvements in Body Composition, Nutritional Intake and Quality of Life in Advanced Cancer Patients on a Phase I Study of a fully Human, Monoclonal Antibody Against the IL-1β.

Patients are given IL-1β-specific IgG mAb intravenously at one of 4 dose levels (0.25, 0.75, 1.25, 3.75 mg/kg) once every 3 weeks. For anorexia-cachexia assessment, serial data on dual-emission X-ray absorptiometry scan (DEXA), bioelectrical impedance analysis (BIA), indirect calorimetry, nutrition diary, and the European Organization for Research and Treatment of Cancer Quality of Life Questionnaire (EORTC QLQ-C30) are utilized. Body composition, nutritional intake and quality of life are compared between baseline and cycle 3.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating cachexia in a human subject, the method comprising the step of administering to the subject a pharmaceutical composition comprising a pharmaceutically acceptable carrier and an amount of an anti-IL-1β Ab effective to reduce at least one symptom of cachexia in the subject.

2. The method of claim 1, wherein the anti-IL-1β Ab is a mAb.

3. The method of claim 2, wherein the mAb is an IgG1.

4. The method of claim 1, wherein the lean body mass of the subject is increased after administration of the pharmaceutical composition.

5. The method of claim 1, wherein the subject's body weight is increased after administration of the pharmaceutical composition.

6. The method of claim 1, wherein the subject's appetite is improved after administration of the pharmaceutical composition.

7. The method of claim 1, wherein the subject has end-stage cancer.

8. The method of claim 1, wherein the subject's muscle mass is increased after administration of the pharmaceutical composition.

9. The method of claim 1, wherein the subject's lean body mass is increased after administration of the pharmaceutical composition.
